(19) United States
(12) Patent Application Publication
(10) Pub. No.: US 2023/0023581 A1
(43) Pub. Date: Jan. 26, 2023

(54) METHOD FOR MEDICAL DETECTION, MEDICAL DETECTION DEVICE, AND STORAGE MEDIUM

(71) Applicant: Jiangyu Kangjian Innovation Medical Technology(Chengdu) Co., Ltd, Chengdu (CN)

(72) Inventors: TAI-CHIEH HUANG, New Taipei (TW); LIANG XIE, Shenzhen (CN); CHIH-MING HUANG, New Taipei (TW)

(21) Appl. No.: 17/863,633

(22) Filed: Jul. 13, 2022

(30) Foreign Application Priority Data
Jul. 14, 2021 (CN) 202110796217.8

(51) Int. Cl. A61B 5/145; A61B 5/00; A61B 5/1486

(52) U.S. Cl. CPC A61B 5/14532; A61B 5/7221; A61B 5/14546; A61B 5/1486; A61B 5/742; A61B 2562/0271

(57) ABSTRACT

A method for medical detection implemented in a medical detection device, includes a detection instruction being received by the device, the device detecting a temperature of a test strip and determining a measurement error rate corresponding to the actual temperature of the test strip, a relationship having been established between temperatures and measurement error rates for test strip temperatures. When the medical detection device receives the test strip that carries blood of a user, at least one physiological parameter based on the blood on the test strip is measured, and a measurement value of the at least one physiological parameter is obtained. And the measurement value is compensated according to the measurement error rate, and a correction value of the physiological parameter is thus obtained.

### FIELD

The subject matter herein generally relates to healthcare, and particularly to a method for medical detection, a medical detection device, and a storage medium.

### BACKGROUND

Medical detection devices are widely used, the medical detection device allows users to measure physiological parameters themselves, so as to reduce the number of times the users go to the hospital. Test strips are usually used while detecting the physiological parameters, for example, the blood glucose meter detects blood glucose of blood on the test strips by electrode measurement. However, the measurement of physiological parameters through the test strips can be easily affected by ambient temperature, which causes measurement errors and affects the accuracy of measurement results.

### BRIEF DESCRIPTION OF THE DRAWINGS

Many aspects of the disclosure can be better understood with reference to the following drawings. The components in the drawings are not necessarily drawn to scale, the emphasis instead being placed upon clearly illustrating the principles of the disclosure. Moreover, in the drawings, like reference numerals designate corresponding parts throughout the several views.

FIG. 1 is a schematic view of an embodiment of a medical detection device according to the present disclosure.

FIG. 2 is a block view of an embodiment of the medical detection device according to the present disclosure.

FIG. 3 illustrates a table of an embodiment of a relationship between temperatures and measurement error rate according to the present disclosure.

FIG. 4 illustrates a flowchart of an embodiment of a method for medical detection according to the present disclosure.

### DETAILED DESCRIPTION

Multiple embodiments are described in the present disclosure, but the description is exemplary rather than limiting, and there may be more embodiments and implementation solutions within the scope of the embodiments described in the present disclosure. Although many possible combinations of features are shown in the drawings and discussed in the detailed description, many other combinations of the disclosed features are also possible. Unless specifically limited, any feature or element of any embodiment may be used in combination with or in place of any other feature or element of any other embodiment.

When describing representative embodiments, the specification may have presented methods and/or processes as a specific sequence of steps. However, to the extent that the method or process does not depend on the specific order of steps described in the present disclosure, the method or process should not be limited to the specific order of steps described. As understood by those of ordinary skills in the art, other orders of steps are also possible. Therefore, the specific order of steps set forth in the specification should not be interpreted as limitation to the claims. In addition, the claims for the method and/or process should not be limited to the steps performed in the written order, and those of skilled in the art may readily understand that these orders may vary and still remain within the essence and scope of the embodiments of the present disclosure.

Unless otherwise defined, technical terms or scientific terms used in the embodiments shall have common meanings as construed by those of ordinary skills in the art to which the present disclosure pertains. The terms “first”, “second” and the like used in the embodiments of the present disclosure do not represent any order, quantity or importance, but are merely used to distinguish among different components. The words “include”, “contain” or the like mean that elements or articles appearing before the words cover elements or articles listed after the words and their equivalents, without excluding other elements or articles. The words “connect”, “link” or the like are not limited to physical or mechanical connection, but may include electrical connection, whether direct or indirect.

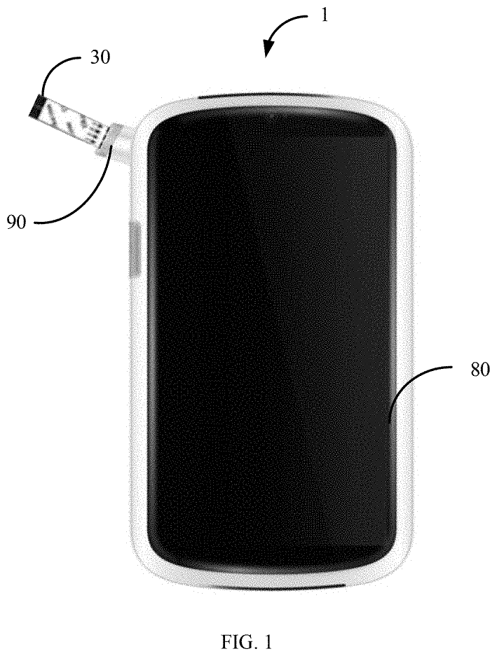

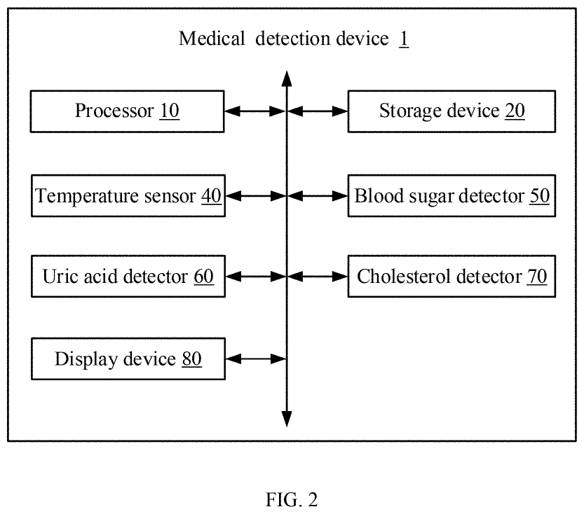

FIGS. 1-2 illustrate an embodiment of a medical detection device 1. The medical detection device 1 includes, but is not limited to, a processor 10, a storage device 20, at least one test strip 30, a temperature sensor 40, a blood sugar detector 50, a uric acid detector 60, a cholesterol detector 70, and a display device 80. FIGS. 1-2 illustrate only one example of the medical detection device 1, other examples can include more or fewer components than as illustrated, or have a different configuration of the various components in other embodiments.

In one embodiment, the storage device 20 can include various types of non-transitory computer-readable storage mediums. For example, the storage device 20 can be an internal storage system, such as a flash memory, a random access memory (RAM) for temporary storage of information, and/or a read-only memory (ROM) for permanent storage of information. The storage device 20 can also be an external storage system, such as a hard disk, a storage card, or a data storage medium. The processor 10 can be a central processing unit (CPU), a microprocessor, or other data processor chip that performs functions of the medical detection device 1.

In response to an instruction generated by user operation, the temperature sensor 40 is controlled to detect the temperature of the test strip 30, the processor 10 is configured to obtain the temperature of the test strip 30 from the temperature sensor 40. The instruction may be a detection instruction for physiological parameters.

In one embodiment, the medical detection device 1 defines a slot 90 for receiving the test strip 30, and the temperature sensor 40 is arranged in the slot 90. The temperature sensor 40 may be an infrared temperature sensor or a contact temperature sensor.

In one embodiment, the display device 80 is a touch screen. The user can input the detection instruction by performing a touch operation on the display device 80, and insert the test strip 30 into the slot 90 before collecting the user's blood using the test strip 30.

In one embodiment, the medical detection device 1 can automatically detect whether the test strip 30 is inserted into the slot 90, and when detecting that the test strip 30 is inserted into the slot 90, the temperature sensor 40 can automatically detect the temperature of the test strip 30. In other embodiments, the user can input a temperature detection instruction by performing a touch operation on the display device 80, and the processor 10 controls the temperature sensor 40 to detect the temperature of the test strip 30 in response to the temperature detection instruction.

The processor 10 is further configured to determine a measurement error rate k corresponding to the detected temperature of the test strip 30 according to a relationship between the temperatures and the measurement error rates of the physiological parameter.

In one embodiment, the storage device 20 stores a table for recording the relationship between the temperatures and the measurement error rates of at least one physiological parameter. The at least one physiological parameter includes blood sugar level, uric acid level, cholesterol level, and other physiological parameter.

For example, when the detection instruction input by the user is a detection instruction of blood sugar, and the temperature sensor 40 detects that the temperature of the test strip is 40° C., it is determined that the measurement error rate of the blood sugar is −8.45% according to the relationship between the temperatures and the measurement error rates.

The processor 10 is further configured to measure at least one physiological parameter based on the blood on the test strip 30, when the medical detection device 1 receives the test strip 30 that carries the blood of the user, that is, the medical detection device receives the test strip and detects presence of blood on the test strip, and obtains the measurement value of the at least one physiological parameter.

In one embodiment, after the temperature of the test strip 30 is detected, the user can use the test strip 30 to collect blood, and insert the test strip 30 that carries the blood into the slot 90. The medical detection device 1 can automatically detect whether the test strip 30 that carries the blood is inserted into the slot 90, and when it is detected that the test strip 30 that carries the blood is inserted into the slot 90, the processor 10 measures the at least one physiological parameter based on the blood on the test strip 30, and obtains the measurement value of the at least one physiological parameter.

In one embodiment, the test strip 30 defines electrodes and enzymes, the enzymes is used for chemically reacting with the glucose, the uric acid, or the cholesterol in the blood.

In detail, the medical detection device 1 can provide various types of test strips, the resistances of the various types of test strips are the same. If the test strip 30 is a strip for blood glucose detection, the enzymes can be glucose dehydrogenase, which can chemically react with the glucose in the blood. If the test strip 30 is a strip for uric acid detection, the enzymes can be uric acid oxidase, which can chemically react with uric acid in the blood. If the test strip 30 is a strip for cholesterol detection, the enzymes can be cholesterol oxidase, which can chemically react with the cholesterol in the blood. Understandably, the test strip 30 can also be set with other enzymes according to other measurable physiological parameters.

In one embodiment, the processor 10 receives signals transmitted by the electrode, the signals generated by the chemical reaction between the enzymes and at least one of the glucose, uric acid, or cholesterol in the blood, and converts the received signals into at least one of the blood glucose concentration, the uric acid concentration, or cholesterol concentration.

In detail, when measuring the blood glucose concentration of the blood of the user, the blood glucose detector 50 receives the signals generated by the chemical reaction between the glucose dehydrogenase and the glucose in the blood, and converts the signals to the blood glucose concentration. When measuring the uric acid concentration of the blood of the user, the uric acid detector 60 receives the signals generated by the chemical reaction between the uric acid oxidase and the uric acid in the blood, and converts the signals to the uric acid concentration. When measuring the cholesterol concentration of the blood of the user, the cholesterol detector 70 receives the signals generated by the chemical reaction between the cholesterol oxidase and the cholesterol in the blood, and converts the received signals into the cholesterol concentration.

The processor 10 is further configured to compensate for the measurement value according to the measurement error rate, and obtain a correction value b of the physiological parameter.

In one embodiment, the correction value b=a−a*k. For example, if the measurement value of the blood glucose concentration is 9 mmol/L, and the measurement error rate is −8.45%, the correction value b is 9.7605 mmol/L.

It should be noted that, the resistance of all test strips is the same. Since the resistance of the test strip will change with the temperature of the environment and the test strip itself, the current received by the medical detector as an indicator will not be accurate, thus affecting the detection results. Therefore, it is necessary to correct the measurement results so as to improve the accuracy of medical conclusions.

The processor 10 is further configured to display the corrected value of the physiological parameter on the display device 80.

In one embodiment, during the production process of the medical detection device 1, the processor 10 is further configured to measure the physiological parameters of different blood samples at different temperatures using a plurality of test strips that carry different blood samples, and obtain a plurality of measurement values of the physiological parameter, establish a table and record the relationship between the measurement values at different temperature and the measurement values of the physiological parameter according to the plurality of measurement values and the different temperatures, and calculate the measurement error rate k corresponding to each temperature according to the average value a1 of the plurality of measurement values corresponding to each temperature and the average value a2 of the plurality of measurement values corresponding to room temperature as a baseline. The measurement error rate k=(a1−a2)/a2, and the relationship between the temperatures and the measurement error rates is stored in the storage device 20.

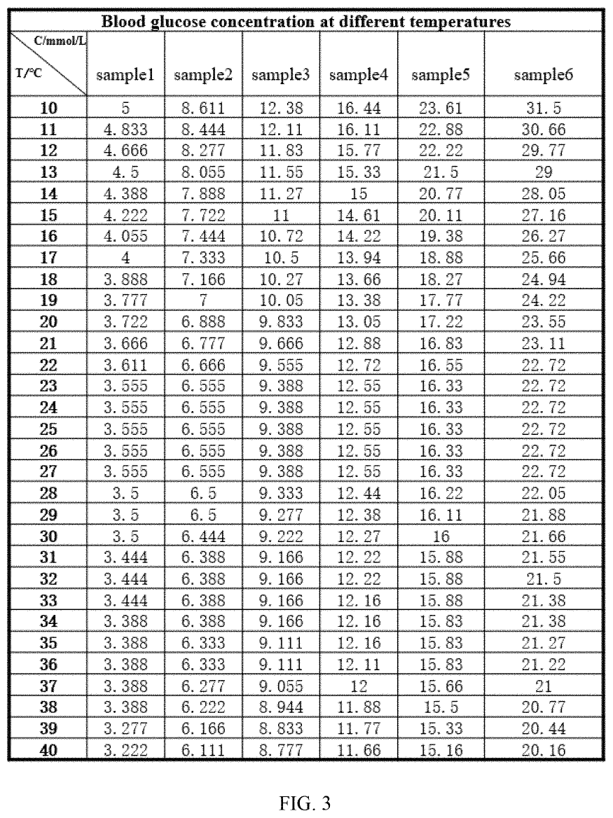

For example, the temperature of the test strip 30 can be controlled by being placed in an incubator, and then the blood glucose concentrations in six samples of blood can be measured. Referring to FIG. 3, it is assumed that the blood glucose concentrations in the blood of the six samples at 40° C. are 3.222 mmol/L, 6.111 mmol/L, 8.777 mmol/L, 11.66 mmol/L, 15.16 mmol/L, 20.16 mmol/L, respectively, and the blood glucose concentrations in the blood of the six samples at room temperature (25° C.) are 3.555 mmol/L, 6.555 mmol/L, 9.388 mmol/L, 12.55 mmol/L, 16.33 mmol/L, and 22.72 mmol/L, respectively. The average value a1 of the plurality of measurement values corresponding to 40° C. is 10.848 mmol/L, and the average value a2 of the plurality of measurement values corresponding to the room temperature is 11.85 mmol/L. Through the above formula, the measurement error rate corresponding to 40° C. can be calculated as −8.45%.

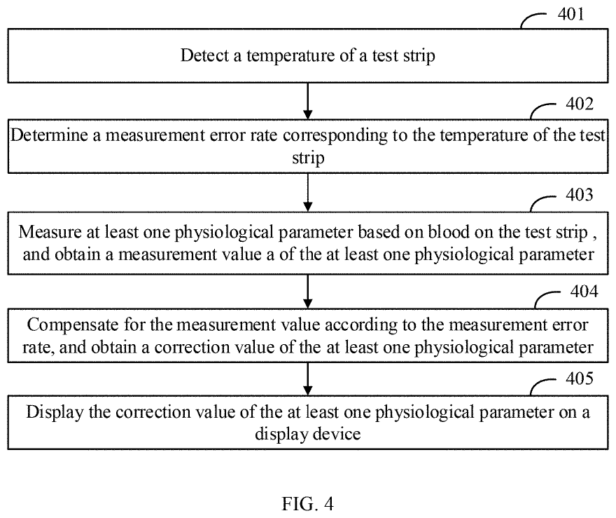

FIG. 4 illustrates the electronic device 1 in one embodiment. The electronic device 1 includes, but is not limited to, a processor 10, a storage device 20, and a computer program 30. FIG. 4 illustrates only one example of the electronic device 1. Other examples can include more or fewer components than as illustrated or have a different configuration of the various components in other embodiments.

The processor 10 can be a central processing unit (CPU), a microprocessor, or other data processor chip that performs functions in the electronic device 1.

FIG. 4 illustrates a flowchart of an embodiment of a medical detection method.

The method is provided by way of example, as there are a variety of ways to carry out the method. The method described below can be carried out using the configurations illustrated in FIGS. 1-2, for example, and various elements of these figures are referenced in explaining the example method. Each block shown in FIG. 4 represents one or more processes, methods, or subroutines carried out in the example method. Furthermore, the illustrated order of blocks is by example only and the order of the blocks can be changed. Additional blocks may be added or fewer blocks may be utilized, without departing from this disclosure. The example method can begin at block 401.

At block 401, in response to a detection instruction generated by a user operation, controlling the temperature sensor 40 to detect the temperature of the test strip 30, and obtaining the temperature of the test strip 30 from the temperature sensor 40.

At block 402, determining a measurement error rate k corresponding to the detected temperature of the test strip, according to a relationship between the temperatures and the measurement error rates of the physiological parameter.

At block 403, measuring the at least one physiological parameter based on the blood on the test strip 30, when the medical detection device 1 receives the test strip 30 that carries the blood of the user, and obtaining a measurement value of the at least one physiological parameter.

At block 404, compensate for the measurement value according to the measurement error rate, and obtain a correction value b of the at least one physiological parameter.

At block 405, display the correction value of the physiological parameter on the display device 80.

Further, the medical detection method includes: measuring the physiological parameters of different blood samples at different temperatures using a plurality of test strips that carry different blood samples, obtaining a plurality of measurement values of the physiological parameter, establishing a table and recording the relationship between the measurement values of the temperature and the measurement values of the physiological parameter in the table according to the plurality of measurement values and the different temperatures, and calculating the measurement error rate k corresponding to each temperature according to the average value a1 of the plurality of measurement values corresponding to each temperature and the average value a2 of the plurality of measurement values corresponding to room temperature as a baseline. The measurement error rate k=(a1−a2)/a2, and the relationship between the temperatures and the measurement error rates is stored in the storage device 20.

It is believed that the present embodiments and their advantages will be understood from the foregoing description, and it will be apparent that various changes may be made thereto without departing from the spirit and scope of the disclosure or sacrificing all of its material advantages, the examples hereinbefore described merely being embodiments of the present disclosure.

What is claimed is:

1. A medical detection device comprising:
at least one processor; and
a storage device coupled to the at least one processor and storing instructions for execution by the at least one processor to cause the at least one processor to:
in response to a detection instruction, control a temperature sensor to detect a temperature of a test strip, and obtain the temperature of the test strip from the temperature sensor;
determine a measurement error rate corresponding to the temperature of the test strip, according to a relationship between temperatures and measurement error rates of physiological parameters;
in response that the medical detection device receives the test strip and detects presence of blood on the test strip, measure at least one physiological parameter based on the blood on the test strip, and obtain a measurement value of each of the at least one physiological parameter; and
compensate for the measurement value according to the measurement error rate, and obtain a correction value of the physiological parameter.

2. The medical detection device according to claim 1, wherein the at least one processor is further caused to:
measure a plurality of physiological parameters of different blood samples from a plurality of test strips at different temperatures, and obtain the measurement value of each of the plurality of the physiological parameters;
establish a table and recording a relationship between temperatures and physiological parameters in the table according to the measurement value of each of the plurality of physiological parameters and the corresponding temperature;
calculate the measurement error rate corresponding to the temperature according to an average value a1 of a plurality of measurement values corresponding to each temperature and an average value a2 of the plurality of measurement values corresponding to different room temperatures, wherein the measurement error rate k=(a1−a2)/a2; and
store the relationship between the temperatures and the measurement error rates in the storage device.

3. The medical detection device according to claim 1, wherein the medical detection device defines a slot, the slot receives the test strip, and the temperature sensor is arranged in the slot, and the temperature sensor is an infrared temperature sensor or a contact temperature sensor.

4. The medical detection device according to claim 1, wherein the at least one physiological parameter comprises at least one of blood sugar level, uric acid level, and cholesterol level.

5. The medical detection device according to claim 1, wherein the medical detection device comprises a plurality of test strips, each of the test strips comprises electrodes and enzymes, the enzymes are designed for chemically reacting with glucose, uric acid, or cholesterol in the blood.

6. The medical detection device according to claim 5, wherein the at least one processor is further caused to:
receive signals generated by a chemical reaction between the enzyme and at least one of the glucose, uric acid, and cholesterol in the blood, the current signal being transmitted by the electrodes; and
convert the signals into at least one of blood glucose concentration, uric acid concentration, or cholesterol concentration.

7. The medical detection device according to claim 1, wherein the at least one processor is further caused to:
display the correction value of the at least one physiological parameter on a display device.

8. A method for medical detection implemented in a medical detection device, comprising:
in response to a detection instruction, controlling a temperature sensor to detect a temperature of a test strip, and obtaining the temperature of the test strip from the temperature sensor;
determining a measurement error rate corresponding to the temperature of the test strip, according to a relationship between temperatures and measurement error rates of physiological parameters;
in response that the medical detection device receives the test strip and detects presence of blood on the test strip, measuring at least one physiological parameter based on the blood on the test strip, and obtaining a measurement value of each of the at least one physiological parameter; and
compensating for the measurement value according to the measurement error rate, and obtaining a correction value of the physiological parameter.

9. The method for medical detection according to claim 8, further comprising:
measuring a plurality of physiological parameters of different blood samples from a plurality of test strips at different temperatures, and obtaining the measurement value of each of the plurality of the physiological parameters;
establishing a table and recording the relationship between the measurement values of the temperature and the measurement values of the physiological parameters in the table according to the measurement value of each of the plurality of physiological parameters and the corresponding temperature;
calculating the measurement error rate corresponding to the temperature according to an average value a1 of a plurality of measurement values corresponding to each temperature and an average value a2 of the plurality of measurement values corresponding to different room temperatures, wherein the measurement error rate k=(a1−a2)/a2; and
storing the relationship between the temperatures and the measurement error rates in the storage device.

10. The method for medical detection according to claim 8, further comprising:
providing a medical detection device comprising a slot for receiving the test strip, and the temperature sensor arranged in the slot, the temperature sensor being an infrared temperature sensor or a contact temperature sensor.

11. The method for medical detection according to claim 8, wherein the at least one physiological parameter comprises at least one of blood sugar concentration, uric acid concentration, and cholesterol concentration.

12. The method for medical detection according to claim 8, further comprising:
providing a plurality of test strips comprising electrodes and enzymes designed for chemically reacting with glucose, uric acid, or cholesterol in the blood.

13. The method for medical detection according to claim 12, wherein measuring at least one physiological parameter based on the blood on the test strip, and obtaining a measurement value of each of the at least one physiological parameter comprises:
receiving signals generated by a chemical reaction between the enzyme and at least one of the glucose, the uric acid, and the cholesterol in the blood, the current signal being transmitted by the electrodes; and
converting the signals into at least one of blood glucose concentration, uric acid concentration, or cholesterol concentration.

14. The method for medical detection according to claim 8, further comprising:
displaying the correction value of the at least one physiological parameter on a display device.

15. A computer-readable storage medium having instructions stored thereon, when the instructions are executed by a processor of a medical detection device, the processor is configured to perform a method for medical detection, wherein the method comprises:
in response to a detection instruction, controlling a temperature sensor to detect a temperature of a test strip, and obtaining the temperature of the test strip from the temperature sensor;
determining a measurement error rate corresponding to the temperature of the test strip, according to a relationship between temperatures and measurement error rates of physiological parameters;
in response that the medical detection device receives the test strip and detects presence of blood on the test strip, measuring at least one physiological parameter based on the blood on the test strip, and obtaining a measurement value of each of the at least one physiological parameter; and
compensating for the measurement value according to the measurement error rate, and obtaining a correction value of the physiological parameter.

16. The storage medium according to claim 15, wherein the method further comprises:
measuring a plurality of physiological parameters of different blood samples from a plurality of test strips at different temperatures, and obtaining the measurement value of each of the plurality of the physiological parameters;
establishing a table and recording the relationship between the measurement values of the temperature and the measurement values of the physiological parameters in the table according to the measurement value of each of the plurality of physiological parameters and the corresponding temperature;
calculating the measurement error rate corresponding to the temperature according to an average value a1 of a plurality of measurement values corresponding to each temperature and an average value a2 of the plurality of measurement values corresponding to different room temperatures, wherein the measurement error rate k=(a1−a2)/a2; and
storing the relationship between the temperatures and the measurement error rates in the storage device.

17. The storage medium according to claim 15, wherein the medical detection device defines a slot, the slot receives the test strip, and the temperature sensor is arranged in the slot, and the temperature sensor is an infrared temperature sensor or a contact temperature sensor.

18. The storage medium according to claim 15, wherein the at least one physiological parameter comprises at least one of blood sugar level, uric acid level, and cholesterol level.

19. The storage medium according to claim 15, wherein the medical detection device comprises a plurality of test strips, each of the test strips comprises electrodes and enzymes, the enzymes are designed for chemically reacting with glucose, uric acid, or cholesterol in the blood.

20. The storage medium according to claim 19, wherein the method further comprises:
receiving signals generated by a chemical reaction between the enzyme and at least one of the glucose, the uric acid, and the cholesterol in the blood, the current signal being transmitted by the electrodes; and
converting the signals into at least one of blood glucose concentration, uric acid concentration, or cholesterol concentration.
